(19) United States
(12) Patent Application Publication
(10) Pub. No.: US 2026/0174339 A1
(43) Pub. Date: Jun. 25, 2026

(54) Fluid-Structure Instability-Based Physiomarker

(71) Applicant: Northwestern University, Evanston, IL (US)

(72) Inventors: Tom Yu ZHAO, Evanston, IL (US); Neelesh A. PATANKAR, Evanston, IL (US); Ethan M. JOHNSON, Chicago, IL (US); Michael MARKL, Chicago, IL (US); Bradley D. ALLEN, Evanston, IL (US); Ben Carlton SMITH, Chicago, IL (US); GUY ELISHA, Chicago, IL (US); Sourav HALDER, Chicago, IL (US)

(21) Appl. No.: 19/129,428

(22) PCT Filed: Nov. 17, 2023
(86) PCT No.: PCT/US23/80380

§ 371 Date: May 13, 2025

Related U.S. Application Data
(60) Provisional application No. 63/384,186, filed on Nov. 17, 2022.

(51) Int. Cl. A61B 5/02; A61B 5/026; G01R 33/563

(52) U.S. Cl. CPC A61B 5/02014; A61B 5/0263; G01R 33/563

(57) ABSTRACT

In certain aspects, a method includes analyzing a single 4D flow magnetic resonance imaging (MRI) scan associated with a blood vessel of a patient, wherein the MRI scan is taken at an initial time point. The method includes determining a critical threshold beyond which an area of the blood vessel in the MRI scan fluctuates unboundedly under infinitesimal perturbations. The method includes selecting a base flow comprising a periodic limit cycle following a pulsatile waveform of blood pressure over a cardiac cycle associated with the blood vessel in the MRI scan. The method includes generating a flutter parameter for describing an onset of an instability triggering fluttering of a vessel wall of the blood vessel in the MRI scan, wherein generating the flutter parameter is based on at least the analyzing of the single 4D flow MRI scan, determining the critical threshold, and the selecting the base flow.

### CROSS REFERENCE TO RELATED APPLICATIONS

The present application claims the benefit of priority under 35 U.S.C. § 119 from U.S. Provisional Patent Application Ser. No. 63/384,186, entitled “FLUID-STRUCTURE INSTABILITY-BASED PHYSIOMARKER,” filed on Nov. 17, 2022, all of which is incorporated herein by reference in its entirety for all purposes.

### TECHNICAL FIELD

The present disclosure generally relates to physiomarkers, and more specifically relates to fluid-structure instability-based physiomarkers.

### BACKGROUND OF THE DISCLOSURE

The physical mechanism that drives aneurysm formation and growth remains fundamentally unresolved. Currently, the clinical diagnosis and treatment of an aneurysm are informed by correlative guidelines on its size and growth rate, along with a holistic consideration of possible symptoms and associated pathologies such as Marfan Syndrome, bicuspid aortic valve, etc. However, aneurysms can exhibit significant growth or rupture before size or rate criteria are met; conversely, large aneurysms may exceed an intervention criterion but nevertheless remain stable over time. Without methods to evaluate the key factors driving aneurysm development, it is difficult to assign preventative treatment such as medical therapy or surgical repair to patients most in need.

The description provided in the background section should not be assumed to be prior art merely because it is mentioned in or associated with the background section. The background section may include information that describes one or more aspects of the subject technology.

### BRIEF DESCRIPTION OF DRAWINGS

The disclosure is better understood with reference to the following drawings and description. The elements in the figures are not necessarily to scale, emphasis instead being placed upon illustrating the principles of the disclosure. Moreover, in the figures, like-referenced numerals may designate to corresponding parts throughout the different views.

FIG. 1 illustrates a distensible blood vessel, modeled as a one-dimensional system with internal pressure P and velocity u averaged across the radial direction r, which is normal to the centerline coordinate x. The interior area A=πR² varies as a function of both space x and time t.

FIG. 2 illustrates a viscous factor β as a function of Womersley number w₀. Here, β has been normalized by its value at w₀=0, corresponding to a parabolic velocity profile.

FIG. 3 illustrates Table 1 depicting that from the basic variable inputs in the first row, the natural length L and time T scales of the system in the middle row are introduced to produce the dimensionless groups in the last row.

FIG. 4 illustrates the marginal stability curve \tilde{μ}=0 as a function of the dimensionless wave number k″ of the perturbation mode and the dimensionless parameter N_{ω}. The dimensionless group N_{ω} encapsulates the blood viscosity, vessel diameter, pressure gradient (or flow acceleration), and viscous contribution under pulsatile waveform of the flow. For a specific value of k″, Nₘ, and \tilde{ω}, N_{ω} within the alternating tongues indicate that the system is unstable to perturbations and can grow unboundedly, whereas N_{ω} outside the tongues correspond to stable base flow. The figure uses representative values of the angular frequency \tilde{ω}=19.6 and Nₘ=1.7×10⁻¹ corresponding to human physiology.

FIG. 5 illustrates Table 2 depicting characteristics of the study cohort, which are summarized for age, sex, as mean±standard deviation and [minimum, maximum] of range or [percentage] values.

FIG. 6A illustrates an example of the maximum sinus of valsalva (SOV) diameter recorded during each clinical visit for one patient. The physiomarker was calculated from an initial MRI taken at year 0.

FIG. 6B illustrates an example of the maximum mid-ascending aorta (MAA) diameter recorded during each clinical visit for one patient.

FIG. 6C illustrates a prediction vs outcome diagram of all patients with follow-up imaging data. The maximum growth rate of their MAA and SOV in (cm/year) are simultaneously visualized by color with respect to theoretical prediction N_{ω,sp}. N_{ω,sp} is calculated from an MRI at time zero. If N_{ω,sp}>0, the patient's marker is labeled by x's.

FIG. 7A illustrates an example of the spatial distribution of the flutter physiomarker N_{ω,sp} for one patient, calculated from their initial MRI taken at year 0. During clinical follow-up, the patient exhibited growth rates of 0.38 cm/year and 0.15 cm/year at their sinus of valsalva (SOV) and mid-ascending aorta (MAA). This agrees with the physiomarker distribution, which shows N_{ω,sp}>0 localized near the SOV rather than the MAA.

FIG. 7B illustrates an example of the spatial distribution of the physiomarker for a second patient, who exhibited growth rates of 0.08 cm/year & 0.30 cm/year at the SOV & MAA. These rates likewise match the physiomarker distribution, where N_{ω,sp}>0 at the MAA rather than the SOV.

FIG. 7C illustrates a prediction vs outcome diagram of all patients with follow-up imaging data. The maximum growth rate of their MAA and SOV in (cm/year) measured from follow-up imaging data are visualized with respect to theoretical prediction N_{ω,sp}, which are measured from a single MRI at time 0. If N_{ω,sp}>0, the patient's marker is labeled by an x. Otherwise, the data point is labeled by a downward pointing triangle. The circles indicate that the patient experienced a surgical intervention after their initial MRI at year 0. The growth threshold of 0.24 cm/year is labeled by black dotted lines. This growth rate optimally discriminates between stable and unstable aneurysms predicted by the proposed physiomarker and falls within the clinically observed range of abnormal growth (0.24 cm/year for small aneurysms to 0.31 cm/year for large aneurysms) that is associated with chronic dissection [16]. The two patients imaged in A and B are marked appropriately.

FIG. 7D illustrates a graph where each patient has been labeled according to whether N_{ω,sp}>0 accurately predicts a growth outcome, categorized as exhibiting a growth rate in SOV or MAA≥0.24 cm/year or experiencing surgical intervention at follow-up.

FIG. 8 illustrates a graph depicting the distribution of the stability parameter N_{ω,sp} in the patient and normal subject cohorts. The median physiomarker value for the normal subject cohort is shown to be significantly (p<0.05) smaller than that for the patient cohort, via a one-tailed Wilcoxon rank sum test.

FIG. 9 illustrates Table 3 where the stability parameter N_{ω,sp} is stratified by age and sex. The one-tailed Wilcoxon rank sum test was used to determine whether the larger median of one population (e.g. patients, Age<40, female) is significantly greater than the smaller median of the other (e.g. normal subjects, Age<40, female). The row of p-values comparing patient and normal subject cohorts is colored red, while the row of p-values comparing sexes is colored blue. The row of p-values comparing each age group is colored green; note that the p-value beneath Age<40 tests the age groups Age<40 and 40≤Age<60, the p-value beneath 40≤Age<60 tests the age groups 40≤Age<60 and 40≤Age, and the p-value beneath Age≥60 tests the age groups Age≥60 and Age<40. Rejection of the null hypothesis at the 5% level is colored orange.

FIG. 10 illustrates Table 4 where each physiological term that contributes to measuring the stability parameter N_{ω,sp} is tabulated for both patients and normal subjects in three age groups. This includes the patient's aorta diameter Aₘ, blood pressure gradient φ̅_{ω} causing oscillatory accelerations, pulsatile contribution to wall shear β_{b}, heartbeat angular frequency ω, and pulse wave velocity c_{pw}. The one-tailed Wilcoxon rank sum test was used to determine whether the larger median of one population (e.g. patients, N_{ω,sp}>0) is significantly greater than the smaller median of the other (e.g. patients, N_{ω,sp}≤0). The row of p-values comparing patient and normal subject cohorts is colored red, while the row of p-values comparing N_{ω,sp}>0 and N_{ω,sp}≤0 is colored blue. Rejection of the null hypothesis at the 5% level is colored orange.

In one or more implementations, not all of the depicted components in each figure may be required, and one or more implementations may include additional components not shown in a figure. Variations in the arrangement and type of the components may be made without departing from the scope of the subject disclosure. Additional components, different components, or fewer components may be utilized within the scope of the subject disclosure.

### DETAILED DESCRIPTION

The detailed description set forth below is intended as a description of various implementations and is not intended to represent the only implementations in which the subject technology may be practiced. As those skilled in the art would realize, the described implementations may be modified in various different ways, all without departing from the scope of the present disclosure. Accordingly, the drawings and description are to be regarded as illustrative in nature and not restrictive.

In certain aspects, the disclosed technology derives an aneurysm physiomarker from first principles. The physiomarker describes a fluid-structure instability that is highly predictive of future aneurysm formation and progression. The physiomarker can be measured for each patient via standard clinical imagining.

In certain aspects, a unifying, ab-intio physiomarker captures physical interactions of known physiological factors, such as, but not limited to, blood pressure, aortic size, wall shear stress, pulse wave velocity, and other appropriate physiological factors, in the progression and development of an aneurysm. Such a physiomarker can be measured for each patient to identify specific regions of a blood vessel that are unstable. This physiomarker can be used to quantify the risk of future abnormal growth and predict aneurysm progression without waiting for a second clinical follow-up years later. No training data or regression analysis needs to be used.

The disclosed technology can be used in many applications including, but not limited to, clinical decision making to identify patients most at risk of abnormal aortic growth, clinical assessment of whether aortic dilatation is in a stable phase or unstable phase (e.g., imminent future pathological growth), pre-surgical planning to determine what segment of aorta must be replaced (e.g., specific blood vessel segment at risk of abnormal dilatation), and other appropriate applications.

The disclosed technology provides improvements upon existing technologies by incorporating fundamental physical fluid-structure analysis of hemodynamic data acquired for standard clinical care.

The disclosed technology provides a fluid-structure instability that may cause blood vessels to dilate and form aneurysms, or induce existing aneurysms to grow significantly. The ab-initio framework yields a physical parameter that describes the onset of linear perturbations as a function of physiological properties such as blood pressure, aortic size, wall shear stress and aortic compliance. The “fluttering” of the aortic wall under this instability mechanism may drive or signal the presence of abnormal aortic growth.

An example study was conducted with 4D flow MRI data from 117 patients indicated for cardiac imaging and 100 normal subjects recruited prospectively as healthy volunteers. Both cohorts were composed of subjects with magnetic resonance imaging (MRI) scans of the full thoracic aorta. A subcohort analysis in 72 patients with at least one subsequent follow-up imaging exam after the initial MRI scan, in which aortic dimensions were assessed by either MRI or computed tomography (CT). The stability parameter was calculated for every subject from the earliest MRI data. For the patient cohort, the growth of each patient's aneurysm was forecast by the stability parameter and compared to the maximum growth rate observed in follow-up imaging and/or any subsequent recorded surgical intervention. A receiving operator characteristic (ROC) curve was generated to gauge the performance of the stability parameter as a potential diagnostic physiomarker. The area under the curve of the ROC analysis was 0.97. No training data was necessary to ‘tune’ the stability parameter. A review of the main factors driving aneurysm evolution revealed two significantly different modes of growth between nascent aortic dilatation in the normal subject cohort and developed aneurysms in the patient cohort.

As discussed in further detail below, the flutter type instability is an important driver of aneurysm growth and formation in many clinical scenarios. It offers accurate prediction of aneurysm development from patient specific data that can inform precise, targeted management of disease progression.

### Introduction

Aneurysms are pathological, localized dilations of a blood vessel that may occur throughout the human body. Intracranial, thoracic aortic, and abdominal aortic aneurysms (IA, TAA, AAA) are each estimated to occur with a global prevalence of 2-5%. Rupture of an aneurysm induces a high rate of mortality and morbidity for the patient. Studies showed that over half of patients with ruptured TAAs or AAAs died before reaching a hospital, with overall mortality ranging from 80 to 100%.

For patients with IA, between 10 to 30% died suddenly away from hospitals, and of those admitted for treatment, 45% experienced an outcome categorized as either moderately disabled, severely disabled, vegetative survival, or death on the Glasgow Outcome Disability Scale. Surgical intervention can be performed to prevent rupture but also carries the risk of complications or death. Thus, it is vital to accurately determine the risk of aneurysm formation and growth to inform timely treatment.

### Current Standard of Care

Generally, the standard of care is to recommend elective treatment for aneurysms based on correlations between rupture risk and aneurysm dimensions. For TAAs, the chance of rupture increases from 2% for diameters between 4 and 4.9 cm to 7% for diameters above 6 cm, while the mean growth rate is approximately 0.1 cm/year. This informs the current clinical practice, which suggests surgical intervention for aneurysm diameters larger than a range between 5.5 to 6.0 cm or exhibiting growth rate larger than a range between 0.5 to 1 cm/year, depending on the aneurysm location and patient history. However, clinical assessment of growth requires comparison between images taken at two time points, typically between 2 to 5 years. Over this period, an aneurysm can grow significantly or rupture fatally. Conversely, an aneurysm which exceeds these empirical criteria may nonetheless remain stable. Thus, prevailing diagnostic guidelines are retrospective and apply population trends to individual patients. To improve predictive capability, the fundamental mechanism underlying aneurysm growth and rupture must be resolved.

### Prediction of Aneurysm Growth and Rupture

The pursuit of a causative relation between aneurysm progression and certain physical properties falling outside a normative range remains inconclusive. For instance, high blood pressure, abnormal wall shear stress distribution, large aortic size, and high wall compliance have all been correlated with aneurysmal growth. However, it is uncertain how these factors interact to trigger abnormal aortic dilatation. For example, high shear stresses have been implicated in some scenarios while low shear stresses in other scenarios.

Thus, prediction of aneurysm growth is based on regression analyses for risk factors such as age or smoking history; regression on morphologic features such as aneurysm diameter or undulation index; machine learning approaches trained on imaging features such as aneurysm diameter or intraluminal thrombi thickness. These traditional methods are based on establishing a correlation between available clinical data and aneurysm growth rates. As with all regression techniques, the breadth of data used to train the model is the main determinant for performance; with a small training cohort relative to the disease population, the predictive capability of the model becomes extrapolative rather than interpolative.

### The Unified Framework of Aneurysm Progression

Here, the subject technology introduces a unifying, ab-intio framework that accounts for the role of known physical factors, such as, for example, blood pressure, aortic size, wall shear stress, and pulse wave velocity, in the development of an aneurysm. The key ansatz is that when these properties fall outside of a normative range, they can trigger a fluid-structure instability that may lead to or signal the onset of abnormal aortic growth.

The dominant properties destabilizing flow within the aorta are the pressure gradient driving blood flow through the blood vessel and the vessel diameter. They cause the vessel wall to ‘flutter’ under higher frequency, oscillatory modes of the heartbeat cycle. Concurrently, the viscous wall shear and wall stiffness stabilize the blood vessel. The competition between these factors reveal an underlying mechanism for aneurysm development as a function of flow and tissue properties. A first principles analysis of these competing factors yield a clinically measurable, dimensionless number that describes the transition from stable flow to unstable aortic fluttering.

A physically intuitive analogy is the fluttering mode of a banner in the wind, where the flow velocity, banner size, drag coefficient, and material elasticity take the place of blood pressure, aortic size, wall shear stress, and pulse wave velocity respectively. Note that the pulse wave velocity, to be formally defined later in the paper, depends on material elasticity. Flutter in this mechanical context induces a significant increase in stresses within the material due to large deformations. The instability that induces aortic wall fluttering may lead to or signal the necessary conditions for aneurysm growth and eventual rupture.

Pulsatile flow in a compliant channel, for example, has been studied, in which the walls of a 2D channel are modeled as spring and damper backed plates. The main instabilities resolved are boundary shear flow instabilities such as the Tollmien-Schlichting wave, which drives the transition to turbulence. Elastic wall deformation is obtained via a Kelvin-Helmholtz type shear instability driven by the Stokes layer near the wall.

Focus is on what mechanisms act on the aortic wall to trigger aneurysm development and progression. The subject technology provides a tubular 1D fluid-structure instability that depends on flow pulsatility, wall shear, blood pressure, and pulse wave velocity (wall stiffness). The wall fluttering stemming from this instability is primarily pressure mediated via the tube law describing the behavior of the elastic tube. Such instability is strongly correlated with abnormal aortic dilatation.

### Application of the Instability-Based Aneurysm Physiomarker

This framework yields a critical threshold beyond which the instability occurs. This criticality condition is obtained from first principles and can be calculated for each patient as a specific parameter encapsulating the instability mechanism potentially driving aneurysm development. This stability parameter is used as a physiomarker to forecast abnormal aortic dilatation.

In a retrospective study of patients indicated for cardiac imaging with follow-up assessment of aortic dimensions available, the proposed flutter physiomarker is highly predictive of whether an aneurysm exhibits abnormal vs natural growth. The only input to calculate the parameter for each patient is a single 4D flow magnetic resonance imaging (MRI) scan taken at an initial time point. This analytical determination was then compared with the clinical outcomes reported from a follow-up at least one year after the baseline MRI to evaluate its potential for predicting significant aortic dilation. As a binary predictor for abnormal growth and surgical intervention, the area under the curve (AUC) for a receiver operating characteristic analysis is 0.997. No training data is necessary to tune the calculation or performance of the physiomarker.

The flutter physiomarker clarifies the exact interaction between physical properties like blood pressure and wall stiffness that affect the instability and associated abnormal growth, and thus what physiological variables must be controlled to prevent this flutter instability. At a macro level, the dominant factor driving aneurysm progression is shown to vary depending on the subjects' aneurysm stage, which is useful for overall disease progression analysis. Patient-level differences are also captured explicitly by the physiomarker, which can show the specific location along the aorta at highest risk for abnormal growth. Lastly, by binning subjects according to age and sex, the proposed physiomarker dominantly describes the clinically observed population traits of aneurysm development in both patient and normal subject cohorts.

### Approach

### Derivation of the Ab-Initio Physiomarker

Here, for example, the subject technology derives the stability parameter from first principles. A classical model for flow through a blood vessel consists of 1D conservation equations for mass and momentum from the Navier-Stokes equations, closed by a constitutive ‘tube law’ for the variation of pressure with the cross-sectional area due to elasticity of the wall. The pressure gradient is chosen to vary periodically in time with frequency equal to that of the heartbeat cycle.

Following this problem formulation in FIG. 1, a stability analysis is conducted to determine a critical threshold beyond which the blood vessel area grows unboundedly under linear perturbations. The blood vessel 10 is assumed to be infinitely long along the axial direction to keep the theoretical analysis tractable. The base flow is chosen to be a periodic limit cycle following the pulsatile waveform of blood pressure over the cardiac cycle. The effect of perturbations at all higher order frequencies are resolved via the Floquet theory. We find that a single dimensionless number describes the onset of the proposed instability which triggers the fluttering of the vessel wall.

### Governing Equations

In 1D, the mass and momentum conservation equations are

$$\frac{\partial A}{\partial t} + \frac{\partial ( uA )}{\partial x} = 0 , \qquad ( 1 )$$

$$\frac{\partial u}{\partial t} + \text{?} Au \frac{\partial u}{\partial x} + - A \frac{\partial P}{\partial x} + 2 π \frac{R}{ρ} {τ}_{w} , \qquad ( 2 )$$

$$\text{?} \text{indicates text missing or illegible when filed}$$

where A[x, t] and R[x, t] denote the cross-sectional area and radius, while the pressure P[x, t] and velocity u[x, t] represent values averaged over the radial profiles at each location x and time t. Here, P is the excess internal pressure inside the blood vessel normalized by the blood density ρ. The wall shear stress term is τ_{w} and \hat{α} is a constant factor that arises from cross-sectional averaging of the non-linear convection term.

To close the problem, the tube law relating pressure to area is taken to be linear

$$P = \frac{{K}_{e}}{ρ} ( \frac{A}{{A}_{o}} - 1 ) \text{?} \qquad ( 3 )$$

$$\text{?} \text{indicates text missing or illegible when filed}$$

where Kₑ is the blood vessel wall stiffness and Aₒ is the relaxed area of the blood vessel corresponding to excess internal pressure P=0. As shown below, the linear stability problem generalizes to any arbitrary tube law relating pressure to vessel area.
Pulse Wave Velocity—Relationship with Aortic Wall Stiffness

The relationship between aortic wall stiffness and pulse wave velocity can be derived by transforming the set of simplified governing equations to the standard form of the wave equation (1). The relevant conservation equations are

$$\frac{\partial A}{\partial t} + \frac{\partial A u}{\partial x} = 0 \text{?} \qquad ( S 1 )$$

$$\frac{\partial u}{\partial t} + u \frac{\partial u}{\partial x} = - \frac{\partial P}{\partial x} , \qquad ( S 2 )$$

$$\text{?} \text{indicates text missing or illegible when filed}$$

where the viscous term has been neglected, and P is the dynamic pressure divided by the blood density. A general tube law is used

$$P - \frac{1}{ρ} G ( A ) , \qquad ( S 3 )$$

where G is some function of the local cross-sectional area. The function G represents the full dependence of the excess internal pressure to the cross-sectional area and thus can encapsulate aortic wall properties such as elastic moduli, wall thickness, etc. in the most general case. Without adding new notation, we next introduce an invertible change in the independent variables x→x+vt and t→t where the the velocity ν is frozen at the mean value. In the new basis, the conservation equations become

$$\frac{\partial A}{\partial t} + A \frac{\partial u}{\partial x} = 0 \text{?} \qquad ( S 4 )$$

$$\frac{{\partial }^{2} u}{\partial t} + \frac{1}{ρ} \frac{dG}{dA} \frac{\partial A}{\partial x} = 0. \qquad ( S 5 )$$

$$\text{?} \text{indicates text missing or illegible when filed}$$

Differentiating the mass equation (eqn. S4) with respect to time and the momentum equation S5) with respect to space gives

$$\frac{{\partial }^{2} A}{\partial {t}^{2}} + A \frac{{\partial }^{2} u}{\partial x \partial t} = 0 , \qquad ( S 6 )$$

$$\frac{{\partial }^{2} u}{\partial x \partial t} + \frac{1}{ρ} \frac{dG}{dA} \frac{{\partial }^{2} A}{\partial {x}^{2}} = 0 , \qquad ( S 7 )$$

which can be combined to obtain

$$\frac{{\partial }^{2} A}{\partial {t}^{2}} - ( \frac{1}{ρ} \frac{dG}{dA} A ) \frac{{\partial }^{2} A}{\partial {x}^{2}} = 0. \qquad ( S 8 )$$

This is the standard form of the wave equation, where the term in parenthesis is typically called the propagation speed. It represents the speed of the plane wave solutions to eqn. S8. The pulse wave velocity can thus be defined as

$${\text{?}}_{pw}^{2} = \frac{1}{ρ} \frac{dG}{dA} A \qquad ( S 9 )$$

$$\text{?} \text{indicates text missing or illegible when filed}$$

### Base Flow

For pulsatile blood flow, the base equilibrium solutions for area A_{b}, pressure gradient P_{b}x, and velocity u_{b} can be written as

$${A}_{b} = {A}_{m} + {A}_{ω} ( k ) ≃ {A}_{m} , \qquad ( 4 )$$

$${u}_{b} = {u}_{m} + {u}_{ω} [ t ] = {u}_{m} + \frac{1}{2} ( {\overline{u}}_{ω} {e}^{i ω t} + {\overline{u}}_{ω}^{*} {e}^{- i ω t} ) , \qquad ( 5 )$$

$$- \frac{\partial {P}_{b}}{\partial x} = {ϕ}_{b} = {ϕ}_{m} + {ϕ}_{ω} [ t ] = {ϕ}_{b} + \frac{{\overline{ϕ}}_{ω}}{2} ( {e}^{i ω t} + {e}^{- i ω t} ) , \qquad ( 6 )$$

where ω is the angular frequency of the heartbeat cycle. Aₘ, uₘ, and φₘ are the temporal mean values of area, velocity, and pressure gradient, respectively. u_{ω} and φ_{ω} are the time dependent, oscillatory components. ū_{ω} is a complex amplitude associated with u_{ω} and superscript * denotes the complex conjugate. The amplitude φ̅_{ω} associated with φ_{ω} is taken to be real for simplicity since it is the driving term. Finally, note that for the given form of the driving pressure gradient

$$\frac{\partial {P}_{b}}{\partial x} ,$$

the pressure P_{b} and consequently the area A_{b} (via the tube law) will vary along the axial (x) direction. Such variations in the base flow are typically on the order of 5% of the mean value as measured via transthoracic echocardiogram.

It assumed, for simplicity, that the area is approximately constant in the base state; that is, A_{b} ≃Aₘ is constant.

The base flow is then described by the conservation equations

$$\frac{\partial {u}_{b}}{\partial x} = 0 , \qquad ( 7 )$$

$${A}_{m} \frac{\partial {u}_{w} [ t ]}{\partial t} = {A}_{m} ( {ϕ}_{m} + {ϕ}_{w} [ t ] ) - {β}_{m} {π𝓋𝓊}_{m} - {β}_{b} {π𝓋𝓊}_{w} [ t ] . \qquad ( 8 )$$

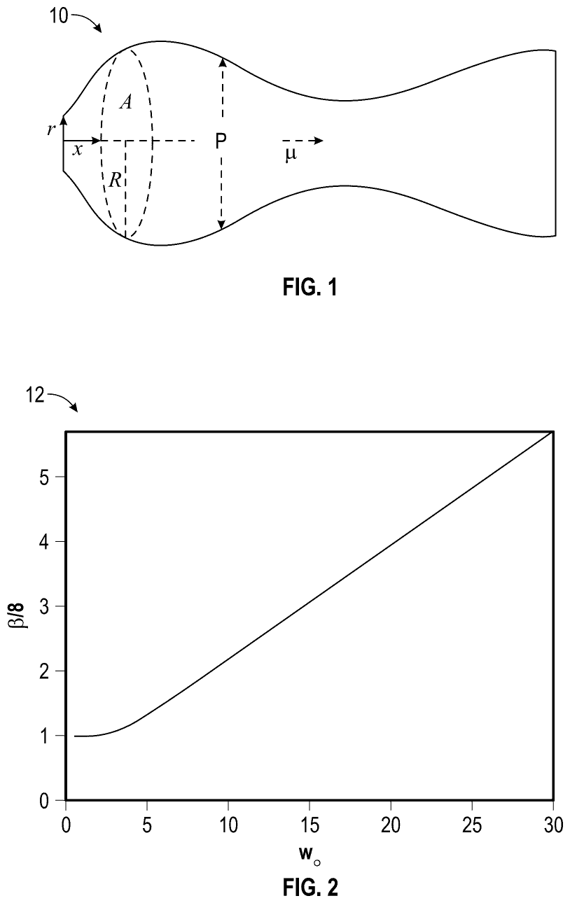

For constant forcing (as opposed to a pulsatile flow), a parabolic velocity profile generates a corresponding wall shear stress

$${τ}_{w , p a r a b o l i c} = \frac{- 4 ρ vu}{R} ,$$

where the kinematic viscosity of blood is given by ν and the negative sign indicates that the wall shear stress on the fluid is pointed in the direction opposite to that of u. For the constant mean flow uₘ we assume parabolic flow based mean shear stress which is equivalent to βₘ=8 in eqn. 8. For the superposed oscillatory flow driven by the heartbeat cycle, the corresponding wall shear stress is obtained from a wall shear coefficient βₚ in the momentum equation via [22].

$${β}_{b} [ {w}_{0} ] = 8 \frac{{τ}_{w}}{{τ}_{w , p a r a b o l i c}} = - 2 {w}_{0} {i}^{3 / 2} ( \frac{{J}_{1} [ {w}_{0} {i}^{3 / 2} ]}{{J}_{0} [ {w}_{0} {i}^{3 / 2} ]} ) ( \frac{1}{1 - 2 \frac{{J}_{1} [ {w}_{0} {i}^{3 / 2} ]}{{J}_{0} [ {w}_{0} {i}^{3 / 2} ]} / ( {w}_{0} {i}^{3 / 2} )} ) . \qquad ( 9 )$$

where Jₙ denote Bessel functions of the first kind. The complex β_{b} represents the ratio of wall shear stress (WSS) at a Womersley number w₀=R\sqrt{w/v}≥0 (pulsatile flow driven at angular frequency ω) to the fully developed WSS associated with w₀=0 (constant forcing). The factor β_{b} is determined via the functional relationship between the wall shear stress τ_{w} on w₀ as derived by Womerlsey (eqn. 9) and displayed in a graph 12 in FIG. 2.

Finally, the mean terms φₘ and uₘ are related through momentum conservation (eqn. 8) via

$${u}_{m} = \frac{{ϕ}_{m} {A}_{m}}{{β}_{m} π 𝓋} .$$

Analogously, the oscillatory flow components are related by

$${{u}^{\text{?}}}_{w} = \frac{{{ϕ}^{\text{?}}}_{w} {A}_{m} ( {β}_{b} π 𝓋 - i ω {A}_{m} )}{{( {β}_{b} π 𝓋 )}^{2} + {( ω {A}_{m} )}^{2}} . \qquad ( 10 )$$

$$\text{?} \text{indicates text missing or illegible when filed}$$

### Linearized Perturbation Equations

Next, the base solutions for the velocity, area, and pressure are perturbed by infinitesimal quantities Y′ of the respective variables,

$$Y = {Y}_{b} + {Y}^{′} = {Y}_{b} + {\sum }_{\text{?} = - ∞}^{∞} {Y}_{k}^{′} [ i ] {e}^{ik \text{?}} , \qquad ( 11 )$$

$$\text{?} \text{indicates text missing or illegible when filed}$$

for Y∈{A, u, P}, where the perturbations Y′ are expressed as the sum of contributions from all wavenumbers k. After linearization of the governing equations and subtracting the base solution, the equations for perturbation components Y′ₖ[t] corresponding to wavenumber k are

$$\frac{\partial {A}_{k}^{′}}{\partial t} + {A}_{b} {iku}_{k}^{′} + {u}_{b} {ikA}_{k}^{′} = 0 , \qquad ( 12 )$$

$${A}_{b} \frac{\partial {u}_{k}^{′}}{\partial t} + {A}_{k}^{′} \frac{\partial {u}_{b}}{\partial t} + {A}_{b} {u}_{b} {iku}_{k}^{′} = - {A}_{b} {ikP}_{k}^{′} - {β}_{b} π {𝓋𝓊}_{k}^{′} + {ϕ}_{b} {A}_{k}^{′} , \qquad ( 13 )$$

$${P}_{k}^{′} = \frac{{K}_{e}}{ρ} \frac{{A}_{k}^{′}}{{A}_{o}} . \qquad ( 14 )$$

We can combine the tube law (eqn. 14) into the momentum equations (eqn. 13) to express pressure P′ₖ in terms of area A′ₖ perturbations. The complex valued solution set tightens to Xₖ=[A′ₖ, u′ₖ]^{T}.

The perturbation equations (eqns. 12, 13) can be written in matrix form as

$${{\underline{X}}_{k}}^{\text{?}} = H {\underline{X}}_{k} , \qquad ( 15 )$$

$$\text{?} \text{indicates text missing or illegible when filed}$$

where Xₖ denotes the time derivative. The vector Xₓ∈C( [symbol] , M_{2,1}[ [symbol] ]), denoting a continuous, functional mapping from a real scalar in time to the complex vector space for [A′ₖ, u′ₖ]^{T}. The coefficient matrix H[t]∈C( [symbol] , M_{2,2}[ [symbol] ]) is periodic with associated frequency ω. This class of periodic linear systems under parametric forcing admits solutions of the Floquet form.

### Floquet Solution

The basis for all solutions to eqn. 15 can be expressed as the product of a periodic component, and an exponential term in time (Theorem 4.1 in Coddington et al.). That is, Xₖ=P(t)e^{Rt}, where P[t] is invertible, P[t]=P[t+2π/ω], and P[t]∈C( [symbol] , M_{2,2}[ [symbol] ]). Xₖ[t]∈C( [symbol] , M_{2,2}[ [symbol] ]) is a complex valued matrix formed from the fundamental solution to eqn. 15. To assess the stability of the solutions Xₖ, we observe that the eigenvalues λ of R determine the stability of the system. Specifically, if there exist λ=μ+iαω such that μ<0 for all wavenumbers k, then the perturbations A′ and u′ decay in time. Otherwise if μ>0 for any wavenumber k, the base solution is unstable, which according to our hypothesis may trigger aneurysm formation and abnormal growth.

To find λ, we see that each solution vector of the fundamental solution takes the form Xₖ=e^{λt}P[t], where P[t] is a vector polynomial with periodic coefficients with associated frequency ω (Section 4.5 in Coddington et al.). This periodic function P[t] can be written as the sum of temporal Fourier modes, such that Xₖ becomes

$${\underline{X}}_{k} = {\sum }_{- ∞}^{∞} {\underline{{X}^{\text{?}}}}_{k , n} {e}^{( μ + i ( n + α ) ω ) t} , \qquad ( 16 )$$

$$\frac{\partial {\underline{X}}_{b}}{\partial t} = {\sum }_{- ∞}^{∞} ( μ + i ( n + α ) ω ) \hat{{\underline{X}}_{k , n}} {e}^{( μ + i ( n + α ) ω ) t} . \qquad ( 17 )$$

$$\text{?} \text{indicates text missing or illegible when filed}$$

Using this in the linearized perturbation equations (eqns. 12, 13), obtains

$$( μ + i ( n + α ) ω ) {\hat{A}}_{k , n} + {A}_{b} ik {\hat{u}}_{k , n} + {u}_{b} {\hat{ikA}}_{k , n} = 0 , \qquad ( 18 )$$

$${A}_{b} ( μ + i ( n + α ) ω ) {\hat{u}}_{k , n} + {\hat{A}}_{k , n} \frac{\partial {u}_{b}}{\partial t} + {A}_{b} {u}_{b} ik {\hat{u}}_{k , n} = - {A}_{b} ik \frac{{K}_{e}}{ρ} \frac{{{A}^{\text{?}}}_{k , n}}{{A}_{o}} - {β}_{b} π v {\hat{u}}_{k , n} + {ϕ}_{b} {{A}^{\text{?}}}_{k , n} . \qquad ( 19 )$$

$$\text{?} \text{indicates text missing or illegible when filed}$$

Note that each equation corresponds to one temporal Fourier mode (nω, where n∈{−∞, ∞}) at a particular spatial wavenumber k. We substitute the base solutions (eqns. 5, 6) to obtain the final homogeneous equation for the Fourier coefficients \hat{X}_{k,n}=[Â_{k,n}, û_{k,n}]^{T}.

$$( μ + i ( n + α ) ω ) {\hat{A}}_{k , n} + {A}_{b} ik {{u}^{\text{?}}}_{k , n} + {u}_{m} {\hat{ikA}}_{k , n} + \frac{1}{2} {\tilde{u}}_{ω} ik {\hat{A}}_{k , n - 1} + \frac{1}{2} {{u}^{\text{?}}}_{ω}^{*} ik {\hat{A}}_{k , n + 1} = 0 , \qquad ( 20 )$$

$${A}_{b} ( μ + i ( n + α ) ω ) {\hat{u}}_{k , n} + {\tilde{u}}_{w} \frac{i ω}{2} {\hat{A}}_{k , n - 1} - {{u}^{\text{?}}}_{ω}^{*} \frac{i ω}{2} {{A}^{\text{?}}}_{k , n + 1} + {u}_{m} {A}_{b} ik {\hat{u}}_{k , n} + \frac{1}{2} {{u}^{\text{?}}}_{ω} {A}_{b} ik {\hat{u}}_{k , n - 1} + \frac{1}{2} {{u}^{\text{?}}}_{ω}^{*} {A}_{b} ik {\hat{u}}_{k , n + 1} = - {A}_{b} ik \frac{{K}_{e}}{ρ} \frac{{\hat{A}}_{k , n}}{{A}_{n}} - {β}_{b} π v {\hat{u}}_{k , n} + {ϕ}_{m} {{A}^{\text{?}}}_{k , n} + \frac{{{ϕ}^{\text{?}}}_{ω}}{2} {\hat{A}}_{k , n - 1} + \frac{{{ϕ}^{\text{?}}}_{ω}}{2} {\hat{A}}_{k , n + 1} , \qquad ( 21 )$$

$$\text{?} \text{indicates text missing or illegible when filed}$$

### Dimensionless Parameters

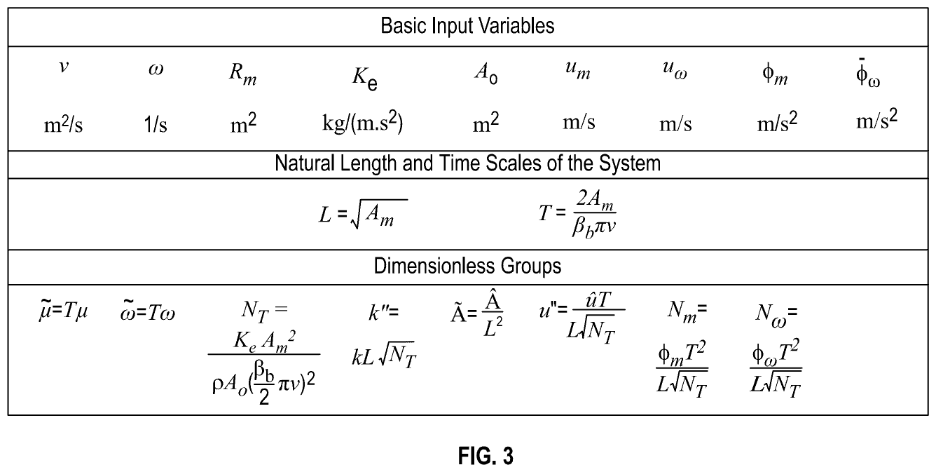

To simplify the representation, the problem is nondimensionalized via Table 1 in FIG. 3. Using the dimensionless groups introduced, the nondimensional forms of the characteristic equations 20 and 21 are

mass conservation equation:

$$( \tilde{μ} + i ( n + α ) \tilde{ω} ) {{A}^{\text{?}}}_{k , n} + {ik}^{″} {u}_{k , n}^{″} + \frac{1}{2} {N}_{m} {ik}^{″} {{A}^{\text{?}}}_{k , n} + \frac{1}{p h [ {β}_{b} ]} \frac{{N}_{ω}}{2 ( 2 + i {ω}^{\text{?}} / ( p h [ {β}_{b} ] ) )} {ik}^{″} {{A}^{\text{?}}}_{k , n - 1} + \frac{1}{p h [ {β}_{b} ]} \frac{{N}_{ω}}{2 ( 2 + i {ω}^{\text{?}} / ( p h [ {β}_{b} ] ) )} {ik}^{″} {{A}^{\text{?}}}_{k , n + 1} = 0 , \qquad ( 22 )$$

$$\text{?} \text{indicates text missing or illegible when filed}$$

### Momentum Conservation Equation:

$$( \tilde{μ} + i ( n + α ) \tilde{ω} ) {u}_{k , n}^{″} + \frac{1}{p h [ {β}_{b} ]} \frac{{N}_{ω}}{2 ( 2 + i {ω}^{\text{?}} / ( p h [ {β}_{b} ] ) )} i {ω}^{\text{?}} {{A}^{\text{?}}}_{k , n - 1} - \frac{1}{p h [ {β}_{b} ]} \frac{{N}_{ω}}{2 ( 2 - i {ω}^{\text{?}} / ( p h [ {β}_{b} ] ) )} {ω}^{\text{?}} {{A}^{\text{?}}}_{k , n + 1} + \frac{{N}_{m}}{2} {ik}^{″} {u}_{k , n}^{″} + \frac{1}{p h [ {β}_{b} ]} \frac{{N}_{ω}}{2 ( 2 + i {ω}^{\text{?}} / ( p h [ {β}_{b} ] ) )} {ik}^{″} {u}_{k , n - 1}^{″} + \frac{1}{p h [ {β}_{b} ]} \frac{{N}_{ω}}{2 ( 2 - i \tilde{ω} / ( p h [ {β}_{b} ] ) )} {ik}^{″} {u}_{k , n + 1}^{″} = - {ik}^{″} {\tilde{A}}_{k , n} - 2 p h [ {β}_{b} ] {u}_{k , n}^{″} + {N}_{m} {{A}^{\text{?}}}_{k , n} \frac{{β}_{m}}{[ {β}_{b} ]} + \frac{{N}_{ω}}{2} {{A}^{\text{?}}}_{k , n - 1} + \frac{{N}_{ω}}{2} {{A}^{\text{?}}}_{k , n + 1} . \qquad ( 23 )$$

$$\text{?} \text{indicates text missing or illegible when filed}$$

where β_{b} is the complex wall shear coefficient as defined earlier (eqn. 9),

$$ph [ {β}_{b} ] = \frac{{β}_{b}}{\vert {β}_{b} \vert}$$

has only the phase information β_{b} due to the normalization by the scalar amplitude |β_{b}|. Finally, \tilde{ω} is the dimensionless angular frequency of the cardiac cycle. The important parameters describing the oscillatory component of flow through the blood vessel—including wall shear coefficient β_{b}, vessel area Aₘ, pressure driven acceleration φ̅_{ω}, and wall stiffness Kₑ—have been collected in a single dimensionless number

$${N}_{ω} = \frac{{\overline{ϕ}}_{ω} {A}_{m}^{1 / 2}}{( \frac{[ {β}_{b} ]}{2} π v )} \sqrt{\frac{ρ {A}_{o}}{{K}_{e}}} . \qquad ( 24 )$$

Akin to the role of the Reynolds number in describing the onset of turbulence, this flutter parameter N_{ω} tracks the inception of the flutter type instability at given values of the remaining parameters. The other nondimensional parameter Nₘ has similar definition as N_{ω} (see Table 1 in FIG. 3) with φₘ replacing φ̅_{ω}. The value of Nₘ is typically smaller (0.05-0.7) compared to the values of N_{ω} (0.5-12) for physiologic conditions. The dimensionless angular frequency \tilde{ω} takes values in the range of 12-34. Finally, the Womersley number w₀ has values in the range of 13-35.

The marginal stability curve \tilde{μ}=0 marks the critical point above which \tilde{μ}>0 perturbation amplitudes grow exponentially in time, and below which \tilde{μ}<0 the base flow is stable under the decay of perturbation modes. To find the locus of points where \tilde{μ}=0, we refer to the methodology proposed by Kumar et al. That is, by fixing the values of k″, \tilde{ω}, Nₘ, and w₀ (and therefore of β_{b}) for a specific flow scenario, as well as presetting \tilde{μ}=0 in eq. 22 and 23, we solve an eigenvalue problem for the critical N_{ω,crit} on the marginal stability curve. This procedure is detailed below.

### Establishing the Marginal Stability Curve

From the characteristic equations 22 and 23, the marginal stability curve where \tilde{μ}=0 can be determined via the method proposed by Kumar et al. The measurable values of k″, \tilde{ω}, and Nₘ are fixed for a specific flow scenario, yielding an eigenvalue problem for the critical N_{ω,threshold} associated with \tilde{μ}=0.

First write the solution set of Fourier coefficients

$${\tilde{A}}_{k , n} = {\tilde{A}}_{k , n}^{r} + i {\tilde{A}}_{k , n}^{i} , {u}_{k , n}^{′′} = {u}_{k , n}^{′′ r} + {iu}_{k , n}^{′′ i}$$

in terms of real and imaginary components. Then the dimensionless characteristic equations 22 and 23 can likewise be separated into purely real and imaginary parts.

### Mass Equation

$${\tilde{A}}_{k , n}^{r} μ + {\tilde{A}}_{k , n}^{i} ( - \ddot{ω} ( α + n ) - \frac{{k}^{′′} {N}_{m}}{2} ) + \frac{{\tilde{A}}_{k , n - 1}^{r} {k}^{′′} {N}_{ω} ( 2 {ph [ {β}_{b} ]}^{′} + \tilde{ω} )}{8 {ph [ {β}_{b} ]}^{r 2} + 2 {( 2 {ph [ {β}_{b} ]}^{i} + \tilde{ω} )}^{2}} + \frac{{\tilde{A}}_{k , n + 1}^{r} {k}^{′′} {N}_{ω} ( 2 {ph [ {β}_{b} ]}^{i} - \tilde{ω} )}{8 {ph [ {β}_{b} ]}^{r 2} + 2 {( \tilde{ω} - 2 {ph [ {β}_{b} ]}^{i} )}^{2}} - \frac{{\tilde{A}}_{k , n - 1}^{r} {ph [ {β}_{b} ]}^{r} + {k}^{′′} {N}_{ω}}{4 {ph [ {β}_{b} ]}^{r 2} + {( 2 {ph [ {β}_{b} ]}^{i} + \tilde{ω} )}^{2}} - \frac{{\tilde{A}}_{k , n + 1}^{i} {ph [ {β}_{b} ]}^{r} + {k}^{′′} {N}_{ω}}{4 {ph [ {β}_{b} ]}^{r 2} + {( \tilde{ω} - 2 {ph [ {β}_{b} ]}^{i} )}^{2}} - {k}^{′′} {u}_{k , n}^{′′ i} + i ( {\tilde{A}}_{k , n}^{r} ( \tilde{ω} ( α + n ) + \frac{{k}^{′′} {N}_{m}}{2} ) + \frac{{\tilde{A}}_{k , n - 1}^{r} {ph [ {β}_{b} ]}^{r} + {k}^{′′} {N}_{ω}}{4 {ph [ {β}_{b} ]}^{r 2} + {( 2 {ph [ {β}_{b} ]}^{i} + \ddot{ω} )}^{2}} + \frac{{\tilde{A}}_{k , n + 1}^{r} {ph [ {β}_{b} ]}^{r} {k}^{′′} {N}_{ω}}{4 {ph [ {β}_{b} ]}^{r 2} + {( \ddot{ω} - 2 {ph [ {β}_{b} ]}^{i} )}^{2}} + {\tilde{A}}_{k , n}^{i} μ + \frac{{\tilde{A}}_{k , n - 1}^{i} {k}^{′′} {N}_{ω} ( 2 {ph [ {β}_{b} ]}^{i} + \ddot{ω} )}{8 {ph [ {β}_{b} ]}^{r 2} + 2 {( 2 {ph [ {β}_{b} ]}^{i} + \ddot{ω} )}^{2}} + \frac{{\tilde{A}}_{k , n + 1}^{i} {k}^{′′} {N}_{ω} ( 2 {ph [ {β}_{b} ]}^{i} - \ddot{ω} )}{8 {ph [ {β}_{b} ]}^{r 2} + 2 {( \ddot{ω} - 2 {ph [ {β}_{b} ]}^{i} )}^{2}} + {k}^{′′} {u}_{k , n}^{′′ r} ) \qquad ( S11 )$$

### Momentum Equation

$$- \frac{{\tilde{A}}_{k , n}^{r} {β}_{m} {N}_{m}}{[ {β}_{b} ]} + {u}_{k , n}^{′′ i} ( - \frac{2 {β}_{p}^{i}}{[ {β}_{b} ]} - \tilde{ω} ( α + n ) - \frac{{k}^{′′} {N}_{m}}{2} ) + {u}_{k , n}^{′′ r} ( \frac{2 {β}_{p}^{r}}{[ {β}_{b} ]} + μ ) - \frac{{\tilde{A}}_{k , n - 1}^{r} {N}_{ω} ( 2 {ph [ {β}_{b} ]}^{r 2} + {ph [ {β}_{b} ]}^{r} ( 2 {ph [ {β}_{b} ]}^{i} + \ddot{ω} ) )}{4 {ph [ {β}_{b} ]}^{r 2} + {( 2 {ph [ {β}_{b} ]}^{i} + \tilde{ω} )}^{2}} + \frac{{\tilde{A}}_{k , n + 1}^{r} {N}_{ω} ( {ph [ {β}_{b} ]}^{r} \tilde{ω} - 2 ( {c}^{2} + {d}^{2} ) )}{4 {ph [ {β}_{b} ]}^{r 2} + {( \tilde{ω} - 2 {ph [ {β}_{b} ]}^{i} )}^{2}} - {\tilde{A}}_{k , n}^{i} k - \frac{{\tilde{A}}_{k , n - 1}^{i} {ph [ {β}_{b} ]}^{r} + {N}_{ω} \tilde{ω}}{4 {ph [ {β}_{b} ]}^{r 2} + {( 2 {ph [ {β}_{b} ]}^{i} + \ddot{ω} )}^{2}} + \frac{{\tilde{A}}_{k , n + 1}^{i} {ph [ {β}_{b} ]}^{r} {N}_{ω} \tilde{ω}}{4 {ph [ {β}_{b} ]}^{r 2} + {( \ddot{ω} - 2 {ph [ {β}_{b} ]}^{i} )}^{2}} + \frac{{k}^{′′} {N}_{ω} {u}_{k , n - 1}^{′′ tr} ( 2 {ph [ {β}_{b} ]}^{i} + \ddot{ω} )}{8 {ph [ {β}_{b} ]}^{r 2} + 2 {( 2 {ph [ {β}_{b} ]}^{i} + \ddot{ω} )}^{2}} + \frac{{k}^{′′} {N}_{ω} {u}_{k , n + 1}^{′′ t} ( 2 {ph [ {β}_{b} ]}^{i} - \tilde{ω} )}{8 {ph [ {β}_{b} ]}^{r 2} + 2 {( \tilde{ω} - 2 {ph [ {β}_{b} ]}^{i} )}^{2}} - \frac{{ph [ {β}_{b} ]}^{r} {k}^{′′} {N}_{ω} {u}_{k , n + 1}^{′′ ti}}{4 {ph [ {β}_{b} ]}^{r 2} + {( \tilde{ω} - 2 {ph [ {β}_{b} ]}^{i} )}^{2}} + i ( {\tilde{A}}_{k , n}^{r} k + {u}_{k , n}^{′ tr} ( \frac{2 {β}_{p}^{i}}{[ {β}_{b} ]} + \ddot{ω} ( α + n ) + \frac{{k}^{′′} {N}_{m}}{2} ) - \frac{{\tilde{A}}_{k , n}^{i} {β}_{m} {N}_{m}}{[ {β}_{b} ]} + {u}_{k , n}^{′′ i} ( \frac{2 {β}_{p}^{r}}{[ {β}_{b} ]} + μ ) + \frac{{\tilde{A}}_{k , n - 1}^{r} {ph [ {β}_{b} ]}^{r} {N}_{ω} \ddot{ω}}{4 {ph [ {β}_{b} ]}^{r 2} + {( 2 {ph [ {β}_{b} ]}^{i} + \tilde{ω} )}^{2}} - \frac{{\tilde{A}}_{k , n + 1}^{r} {ph [ {β}_{b} ]}^{r} {N}_{ω} \ddot{ω}}{4 {ph [ {β}_{b} ]}^{r 2} + {( \ddot{ω} - 2 {ph [ {β}_{b} ]}^{i} )}^{2}} - \frac{{\tilde{A}}_{k , n - 1}^{r} {N}_{ω} ( 2 {ph [ {β}_{b} ]}^{r 2} + {ph [ {β}_{b} ]}^{i} ( 2 {ph [ {β}_{b} ]}^{i} + \tilde{ω} ) )}{4 {ph [ {β}_{b} ]}^{r 2} + {( 2 {ph [ {β}_{b} ]}^{i} + \tilde{ω} )}^{2}} + \frac{{\tilde{A}}_{k , n + 1}^{r} {N}_{ω} ( {ph [ {β}_{b} ]}^{i} \tilde{ω} - 2 ( {c}^{2} + {d}^{2} ) )}{4 {ph [ {β}_{b} ]}^{r 2} + {( \tilde{ω} - 2 {ph [ {β}_{b} ]}^{i} )}^{2}} + \frac{{ph [ {β}_{b} ]}^{r} {k}^{′′} {N}_{ω} {u}_{k , n - 1}^{′′ r}}{4 {ph [ {β}_{b} ]}^{r 2} + {( 2 {ph [ {β}_{b} ]}^{i} + \ddot{ω} )}^{2}} + \frac{{ph [ {β}_{b} ]}^{r} {k}^{′′} {N}_{ω} {u}_{k , n + 1}^{′′ r}}{4 {ph [ {β}_{b} ]}^{r 2} + {( \ddot{ω} - 2 {ph [ {β}_{b} ]}^{i} )}^{2}} + \frac{{k}^{′′} {N}_{ω} {u}_{k , n - 1}^{′′ i} ( 2 {ph [ {β}_{b} ]}^{i} + \tilde{ω} )}{8 {ph [ {β}_{b} ]}^{r 2} + 2 {( 2 {ph [ {β}_{b} ]}^{i} + \tilde{ω} )}^{2}} + \frac{{k}^{′′} {N}_{ω} {u}_{k , n + 1}^{′′ i} ( 2 {ph [ {β}_{b} ]}^{i} - \tilde{ω} )}{8 {ph [ {β}_{b} ]}^{r 2} + 2 {( \tilde{ω} - 2 {ph [ {β}_{b} ]}^{i} )}^{2}} ) = 0 \qquad ( S12 )$$

where

$$ph [ {β}_{b} ] = {ph [ {β}_{b} ]}^{r} + {i ph [ {β}_{b} ]}^{i} and {β}_{b} = {β}_{b}^{r} + i {β}_{b}^{i} .$$

The real and imaginary parts of eqn. S11 as well as S12 must each be identically zero to fully satisfy both characteristic equations. This provides four equations for four unknowns,

$$\underline{Z} = {[ {\tilde{A}}_{k , n}^{r} , {\tilde{A}}_{k , n}^{i} , {u}_{k , n}^{′′ r} , {u}_{k , n}^{′′ i} ]}^{′} ,$$

which can be written as a linear equation JZ=0. The matrix J thus comprises the linear coefficients of Z in the real & imaginary parts of the mass (eqn. S11) and momentum (eqn. S12) equations.

The real part of the temporal growth rate is set to \tilde{μ}=0. The imaginary part takes on the value α=½ for the subharmonic resonance in which the response frequency is half the driving frequency, and α=0 for the harmonic case where the response frequency is the same as the driving frequency \tilde{ω}. Since integer multiples of \tilde{ω} can be absorbed into the periodic function P(t), α is defined modulo \tilde{ω}. The Fourier terms with 0<α<½ is equivalent to the complex conjugate terms associated with ½<α<1, so consideration can be restricted to 0≤α≤½. However, we note that 0<α<½ are only associated with stable flows \tilde{μ}≤0 in the range of physiologically viable N_{ω}.

The critical N_{ω,crit} corresponding to the marginal stability curve \tilde{μ}=0 as well as the k″, \tilde{ω}, and Nₘ selected for a specific flow scenario can be found by solving an eigenvalue problem. Specifically, we decompose J into C, the linear coefficients of Z in J that do not contain N_{ω}, and D, the entries of J that are proportional to N_{ω}. The linear matrix equation becomes

$$C \underline{Z} + {N}_{ω} D \underline{Z} = 0. \qquad ( S 13 )$$

This can be written as an eigenvalue problem

$$- inv ( C ) D \underline{Z} = \frac{1}{{N}_{ω , crit}} \underline{Z} . \qquad ( S 14 )$$

where the eigenvalues of the matrix −inv(C)D are reciprocals of the critical N_{ω,crit} on the marginal stability curve. The preset values of \tilde{ω} and Nₘ are measured from patient MRI, and k″ is swept through to obtain the “tongues” observed in FIG. 2. The minimum critical N_{ω} associated with the first subharmonic tongue to appear as k″ increases is chosen as the threshold N_{ω,threshold}. It is the global minimum of N_{ω,crit} on all marginal stability tongues, such that the flutter instability is triggered first for increasing N_{ω} at the value N_{ω,threshold}.

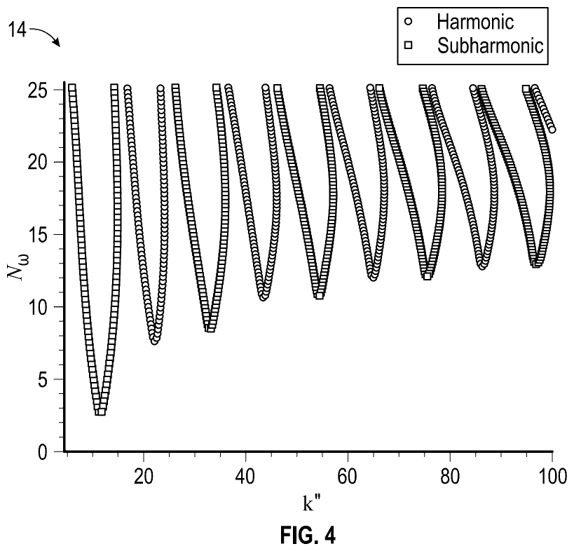

In a graph 14 in FIG. 4, the harmonic α=1 and subharmonic α=½ “tongues” of instability are plotted. The space of dimensionless wavenumber k″ and flutter parameter N_{ω} is divided into tongue regions of instability, where perturbations to the flow grow in time, and outside zones of stability, where the base flow remains stable to perturbations. The harmonic response has the same frequency \tilde{ω} (and its multiples) as that of the driving pressure gradient in the base flow whereas the subharmonic response has half the frequency \tilde{ω} (and its multiples). The subharmonic solution is excited first as No increases past the lowest threshold value N_{ω,threshold}. This threshold value occurs at the bottom tip of the first subharmonic tongue and is the global minimum of the critical flutter parameter on all tongues of marginal stability, min(N_{ω,crit})=N_{ω,threshold} (see FIG. 4).

If N_{ω}>N_{ω,threshold}, the blood vessel will be unstable to a waveband of perturbation modes, whereas below this threshold, the base flow should remain stable. The growth of perturbation modes will trigger or signal the permanent dilatation of a cross-sectional area of the blood vessel over time. The flutter parameter may be tested to determine whether it is predictive of future aortic growth and potential aneurysm development by measuring the patient specific physiological properties comprising N_{ω,clin} (e.g., through cardiac imaging) and validating this theoretical forecast against observed aortic dilatation at follow-up.

### Pulse Wave Velocity

To determine the flow stability for a specific patient, the above formulation requires information about the wall stiffness Kₑ of the blood vessel. This physiological property can be found from the pulse wave velocity (PWV) measured from imaging techniques such as MRI scans and echocardiograms. The PWV is the propagation speed of the pulse wave in the aorta and is related to the elastic modulus or stiffness of the aortic wall. This relationship can be derived by transforming the set of simplified governing equations to the standard form of the wave equation [1]. The relevant conservation equations are discussed above with respect to equations S1-S9.

Although a linear tube law was used in the derivation of the flutter parameter, a general tube law is likewise permissible via

$$P = \frac{1}{ρ} G ( A ) ,$$

where G can be some nonlinear function of the local cross-sectional area. The function G represents the full dependence of the excess internal pressure on the cross-sectional area and thus can encapsulate aortic wall properties such as elastic moduli, wall thickness, etc. in the most general case. The pulse wave velocity can then be shown as

$${c}_{p ω}^{2} = \frac{1}{ρ} \frac{dG}{dA} A . \qquad ( 25 )$$

In the context of the flutter parameter derivation, the tube law term appears in the linearized perturbation equations. By expansion around the base pressure P_{b} and area Aₘ,

$$P = {P}_{b} + {P}^{′} = {P}_{b} + \frac{1}{ρ} \frac{dG}{dA} {\vert}_{b} {A}^{′} = {P}_{b} + {c}_{p ω}^{2} \frac{{A}^{′}}{{A}_{m}} . \qquad ( 26 )$$

It can be seen that no matter which form the tube law G(A) takes, the measured PWV can be used to quantify the blood vessel's elastic properties. The key dimensionless parameter can be recast in terms of PWV as follows

$${N}_{ω} = \frac{{\overline{ϕ}}_{ω} {A}_{m}^{1 / 2}}{( \frac{\vert {β}_{b} \vert}{2} π v )} \sqrt{\frac{ρ {A}_{o}}{{K}_{e}}} = \frac{{\overline{ϕ}}_{ω} {A}_{m}}{\frac{\vert {β}_{b} \vert}{2} π v {c}_{p ω}} . \qquad ( 27 )$$

Using eqn. 27, N_{ω} can now be calculated explicitly from clinical imaging data for each cross-section along a blood vessel 10. The difference between this clinical patient specific value N_{ω,clin} and the critical threshold N_{ω,threshold} on the marginal stability curve, produces an overall stability parameter

$${N}_{ω , sp} [ {N}_{m} , \ddot{ω} , {β}_{b} [ {ω}_{0} ] ] = {N}_{ω , clin} - {N}_{ω , threshold} [ {N}_{m} , \tilde{ω} , {β}_{b} [ {ω}_{0} ] ] . \qquad ( 28 )$$

Eqns. 22 and 23 imply that N_{ω,threshold} depends on Nₘ, \tilde{ω}, and β_{b}. This is reflected in eqn. 28 from the functional dependence of N_{ω,threshold} and by consequence of N_{ω,sp} on these parameters. Note that β_{b} depends on the Womersley number w₀ (eqn. 9). All the independent parameters (Nₘ, \tilde{ω}, w₀) can be determined clinically.

If N_{ω,sp}>0, the blood vessel cross-section is expected to grow due to the increase in perturbation amplitude. Otherwise for N_{ω,sp}≤0, the blood vessel diameter should remain constant in time since all perturbation modes decay. Thus, the stability parameter N_{ω,sp} can serve as a predictive physiomarker of abnormal aortic growth and is convenient to apply clinically.

In summary, an ab-initio theoretical framework is provided to predict the stability of an aortic section depending on a patient's aorta diameter Aₘ, blood pressure gradient φ̅_{ω} causing oscillatory acceleration, pulsatile contribution to wall shear β_{b}, blood viscosity ν, and blood density ρ. These values can be extracted from 4D flow MRI.

### Cohort Selection

To gauge the performance of the flutter physiomarker in analyzing aneurysm growth, a retrospective study was carried out for patients with and without existing aortopathies. A database of patients indicated for clinical cardiac imaging, including 4D flow MRI, at Northwestern Memorial Hospital between 2011 and 2019 was queried to identify a list of subjects with suspected isolated aortopathy and normal, tricuspid aortic valve (TAV). Exclusion criteria were aortic valve stenosis (mild to severe), ejection fraction lower than 50%, bicuspid aortic valve, history of aortic dissection, or history of valve replacement or aortic repair. Subjects both with and without aortic dilatation—clinical measurement of maximal-area ascending aortic (MAA) or sinus of valsalva (SOV) diameter greater or equal to 4 cm—were included.

Patients in the initial cohort formation with genetic tissue disorders, congenital heart malformations, or history of aortic or mitral valve repair were not excluded from analysis. However, for evaluation of predictive performance of N_{ω} (or equivalently N_{ω,sp}), a subcohort of patients without concurrent disease and isolated aortopathy was created.

An additional group of healthy subjects was assembled as a normal subject cohort for comparison. These subjects were drawn from a separate database of prospectively-recruited healthy volunteers who received 4D flow MRI. Subjects eligible for enrollment were 18 and older and had no known cardiovascular disease or abnormalities. The cohort for analysis was chosen from the overall group of recruited volunteers to be uniformly distributed by age and sex.

All subjects were included in this study with oversight by and approval from the Northwestern University Institutional Review Board. Patients were enrolled by retrospective chart review and waiver of consent. Healthy subjects were enrolled with prospectively obtained informed consent.

### Clinical Chart Reviews and Patient Outcomes Classification

Clinical patient records were reviewed comprehensively for the isolated aortopathy cohort to identify the occurrence or lack of aortic diameter growth or aortic surgery after the 4D flow imaging for each patient. Aortic diameter growth was assessed from radiological measurements taken with CT or MR angiography imaging, which included standardized assessment of MAA and SOV diameter in double-oblique view. Aortic surgery events included any surgical replacement or repair of the aortic valve or any portion of the aorta between sinus and arch. Times between 4D flow imaging and follow-up measurements or surgical events were noted.

Growth events were classified by maximum rate of change over any sequential follow-up imaging, e.g., evolution of SOVₘₐₓ is characterized by the maximum difference rate through follow-up time:

$${max}_{∀ t} ( \frac{Δ {SOV}_{max}}{Δ t} ) .$$

This is representative of clinical decision making that identifies potential need for elective surgical intervation from observing interval change in aortic diameter.

### Cohort Characteristics

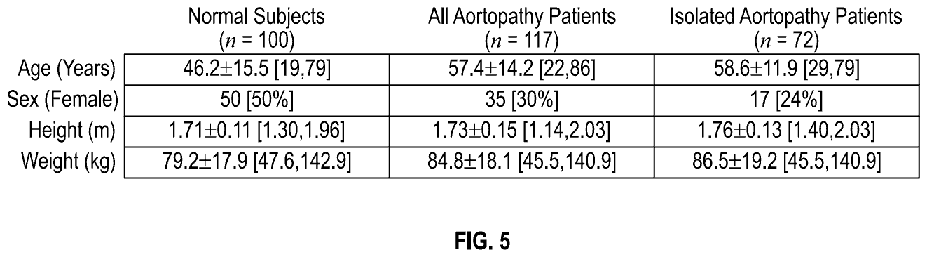

From the database of subjects with clinically indicated cardiac and 4D flow MRI, a total of 125 patients with suspected aortopathy and normal TAV were identified for potential inclusion in this study. Of the patients identified, 8 patients were excluded due to imaging artifacts in their 4D flow MRI, resulting in a cohort of 117 patients with suspected aortopathy. In the subcohort of isolated aortopathy patients, a total of 72 patients had at least one angiography exam occurring subsequent to the 4D flow imaging analyzed here. Additionally, a total of 100 healthy control subjects were identified, and the selections represented a wide range of ages and sexes in the cohort, with subjects aged 19 years to 79 years and 50% of the group female. The cohort demographic characteristic statistics are summarized in Table 2 in FIG. 5.

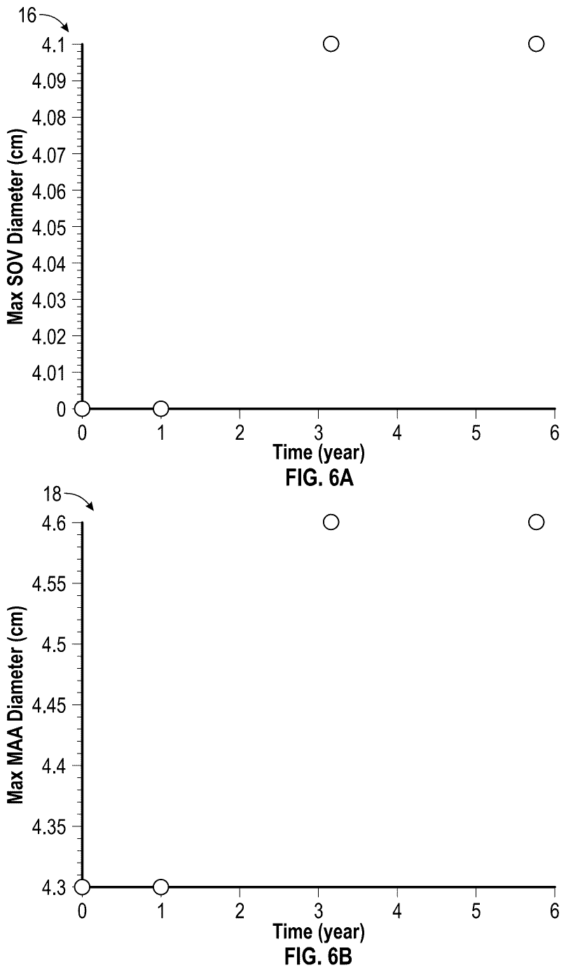

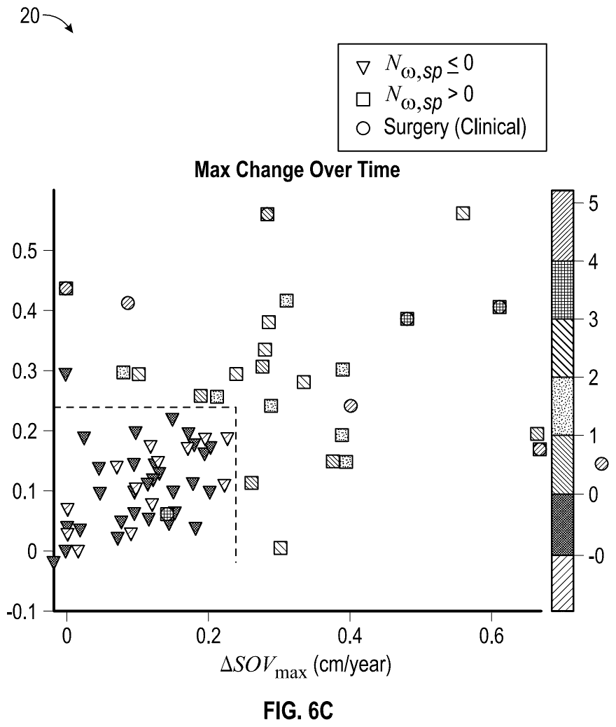

A total of 72 patients in the patient cohort had at least one clinical follow-up visit, allowing growth rate to be quantified, and had no exclusions found in chart review (history of congenital heart malformations, connective tissue disorder or prior valve operations). The maximum of their SOV and MAA diameters (SOVₘₐₓ and MAAₘₐₓ) recorded during each clinic visit are presented as a time series after their initial MRI at year 0 (FIGS. 6A and 6B). An example of patient time-series aortic dimensions is shown in a graph 16 FIG. 6A, ΔSOVₘₐₓ=0.05 cm/year due to a stepwise jump in measured SOV max between years 2 to 3. This growth rate is defined analogously for the maximum MAA diameter; a graph 18 in FIG. 6B gives ΔMAAₘₐₓ=0.14 cm/year for the same patient.

FIG. 6C illustrates a prediction vs outcome diagram 20 of all patients with follow-up imaging data. The maximum growth rate of their MAA and SOV in (cm/year) are simultaneously visualized by color with respect to theoretical prediction N_{ω,sp}. N_{ω,sp} is calculated from an MRI at time zero. If N_{ω,sp}>0, the patient's marker is labeled by x's. Otherwise, the data point is labeled by downward pointing triangles. The circles indicate that the patient experienced a surgical intervention after their initial MRI at year 0. N_{ω,sp}>0 appears to correlate with larger growth rates for the MAA and SOV. The growth threshold of 0.24 cm/year is labeled by black dotted lines—this value is outside the normal range of growth in TAAs of all sizes (<0.2 cm/year) and optimally discriminates between stable and unstable aneurysms predicted by the proposed physiomarker. This optimal threshold of 0.24 cm/year falls within the clinically observed range of abnormal growth (0.24 cm/year for small aneurysms to 0.31 cm/year or large aneurysms) that is associated with chronic dissection.

### Results

### Patient Aortic Growth and N_{ω,Sp} Predictive Performance

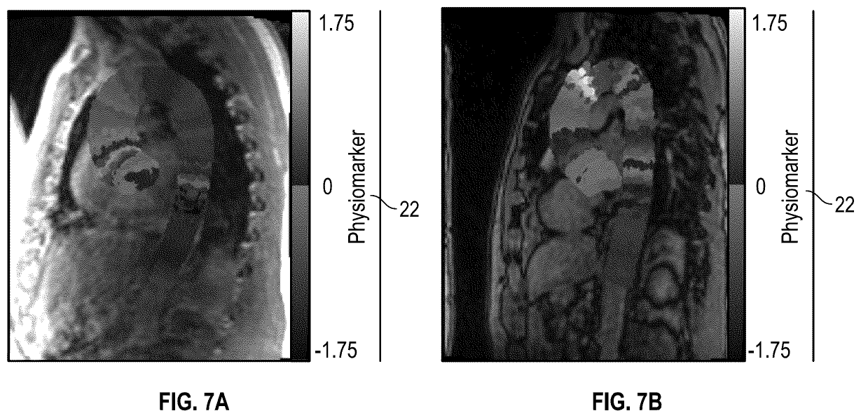

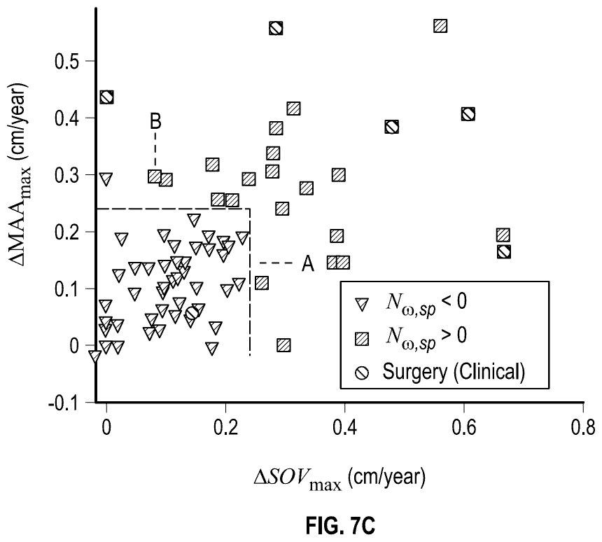

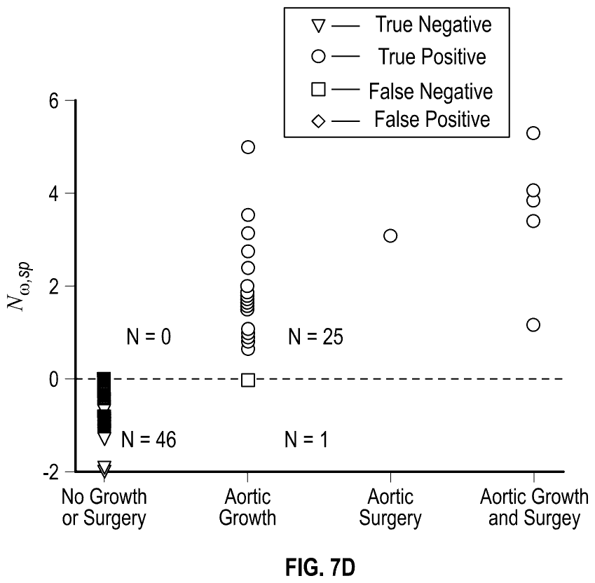

Since the flow velocities are resolved spatially through 4D-flow MRI, the physiomarker 22 (N_{ω,sp}) can be visualized based on location along the centerline of the aorta as a result of our 1D analysis (FIGS. 7A-7D). The physiomarker 22 is calculated from the patient's initial MRI taken at year 0, and can be evaluated against follow-up data that report SOV and MAA diameters. For instance, FIG. 7A shows that a patient's growth rates of 0.38 cm/year and 0.15 cm/year at their SOV and MAA respectively agrees with their spatial physiomarker distribution, which exhibits N_{ω,sp}>0 localized near the SOV rather than the MAA. Similarly, FIG. 7B demonstrates a match between a second patient's growth rates of 0.08 cm/year & 0.30 cm/year at the SOV & MAA and their physiomarker 22 distribution exhibiting N_{ω,sp}>0 at the MAA rather than the SOV.

Next, the per patient growth rates for the SOV and MAA are visualized in FIG. 7C and compared with our theoretical predictions. Each “×” in FIG. 7C denotes N_{ω,sp}>0, as calculated from the patient's MRI image at year 0. This indicates that the ascending aorta is expected to grow due to the flutter type instability. Conversely, each “∇” represents N_{ω,sp}≤0. Since all perturbation modes are damped in this case, the ascending aorta should not be subject to the identified instability. Data points for patients who experienced surgical intervention after their initial MRI are circled. All physiomarker 22 values N_{ω,sp} were calculated from patient MRI at time zero, without reference to follow-up data.

Growth rates exceeding 0.2 cm/year lie outside the range of normal growth of the thoracic aorta. When the stability of aneurysm measured at time zero via N_{ω,sp} are plotted with respect to the growth rates measured from follow-up data, we find that a growth threshold of 0.24 cm/year optimally discriminates between stable and unstable aneurysms forecast by the proposed physiomarker 22. This is an emergent division of the growth data based purely on the transition of N_{ω,sp} from negative to positive—from natural aortic dilatation over time to abnormal growth driven by unstable flutter. Thus, the theoretical boundary filters out growth associated with the proposed instability.

The proposed optimal boundary of 0.24 cm/year falls within the clinically observed range of significantly higher growth rates (0.24 cm/year for smaller 4 cm aneurysms to 0.31 cm/year or larger 5.2 cm aneurysms) that is associated with chronic dissection in patients. This agreement between the theoretical boundary based on the flutter physiomarker 22 and the statistically significant growth rate that clinicians have independently deduced validates the physiomarker 22 as an unbiased, clinically valuable predictor of abnormal aneurysm growth. This boundary for abnormal growth has been visualized in FIG. 7C.

FIG. 7D shows that by using the aortic growth rate of 0.24 cm/year as an indicator of significant growth, the stability parameter N_{ω,sp}>0 serves as a good binary predictor for the growth outcome of each patient. The accuracy, sensitivity, and specificity of this proposed physiomarker 22 in predicting abnormal growth in the thoracic aorta are 0.986, 0.962, and 1.000, respectively. The area under the curve (AUC) of a receiver operating characteristic (ROC) analysis is 0.997; an AUC above 0.9 is typically considered “outstanding” for the performance of a binary predictive diagnostic.

Additionally, the optimal operating point occurs at the minimum positive value for N_{ω,sp} for patients with follow-up data, suggesting that the analytically derived threshold N_{ω,threshold} accurately describes the onset of the underlying instability. No training data set was necessary to tune the calculation of the physiomarker 22 for each patient. If the more conservative threshold 0.31 cm/year is selected instead as a binary indicator of clinically significant growth, the accuracy, sensitivity, specificity, and AUC of this proposed physiomarker become 0.875, 1.000, 0.8393, and 0.952 respectively. Its performance in classifying abnormal growth is therefore bounded from below in the “outstanding” category for typical clinical use cases.

### Cohort Comparisons

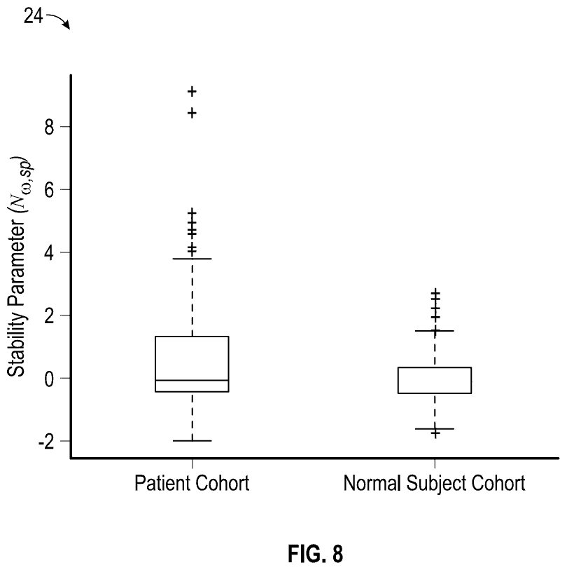

Next, the distributions of the stability parameter N_{ω,sp} in both the normal subject cohort and the patient cohort are examined. As seen in a diagram 24 in FIG. 8, the median physiomarker value for the normal subject cohort is shown to be significantly (p=0.0370) smaller than that for the patient cohort, via a one-tailed Wilcoxon rank sum test. This agrees with the inclusion criteria used to establish the patient and normal subject cohorts, as FIGS. 6A-6C shows that the physiomarker N_{ω} appears to trend with increased growth rates in the SOV and MAA. The sample size of both cohorts exceed 93, the value required to establish significance at a level of p<0.05 for the difference in their median values with 90% statistical power.

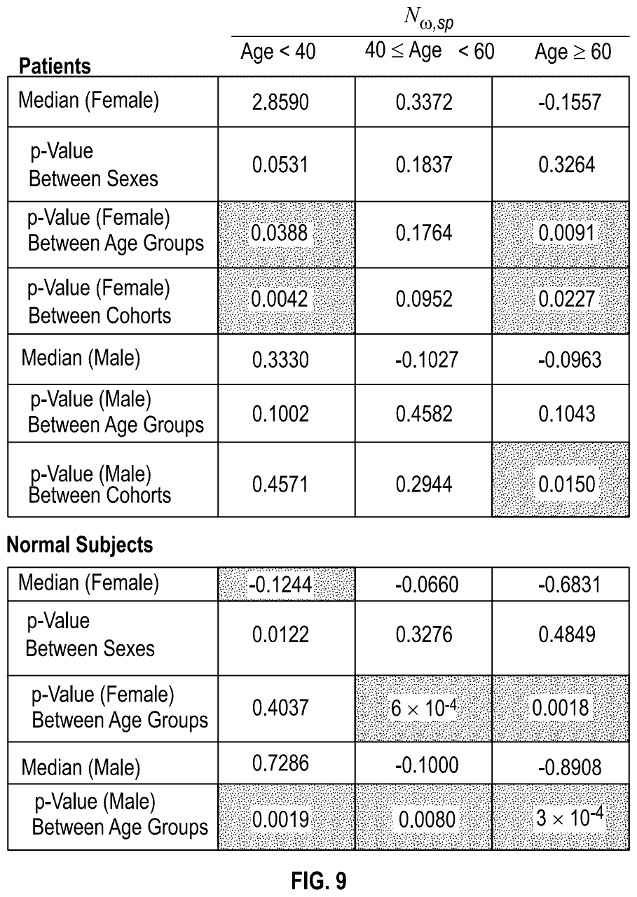

The physiomarker 22 measured for patient and normal subject cohorts into different age and sex groups was binned in Table 3 in FIG. 9. The female normal subjects in the youngest age group (Age<40) show a significantly smaller physiomarker value compared to males in the same cohort. This could reflect population level observations that TAAs occur more commonly in males than in females, despite poorer outcomes in females. We also note that for the patient cohort, females exhibit systematically though not significantly higher N_{ω,sp} than males across every age group. This sex disparity may mirror clinical observation that TAA growth is accelerated in females compared to males. Thus, the distribution of the physiomarker 22 among different sex and age groups in the two cohorts appear to agree with general population trends reported in the literature.

### Discussion

### Predictive Power of the Physiomarker

While the physiomarker 22 proves predictive of abnormal growth, it is not expected to discriminate between no growth and any nonzero amount of aortic dilatation. After all, growth in aortic dimensions occurs with natural aging. Normal aortic growth, while complex in etiology, is generally understood to occur as a result of the repeated natural stresses induced by pressurized blood flow, which ultimately result in gradual loss of elastin fibers, remodeling of elastic lamellae, and ultimately increase of vessel diameter. Normative rates of increase in adults have been assessed at 0.11 cm/year in adults for both men and women, but several factors such as blood pressure and body surface area are associated with larger diameters, and there is high variation in baseline aortic diameter at any given age range.

Instead of describing all modes of aortic dilatation, the physiomarker 22 identifies the specific presence of the flutter instability, which appears to signal subsequent abnormal growth for a significant percentage of patients who eventually experience rates exceeding 0.24 cm/year. Thus, the physiomarker's ability to predict abnormal dilatation in contrast to natural growth is crucial for prompt clinical decision-making and accurate treatment.

The physiomarker 22 may also expand aneurysm detection and prediction to aortic segments heretofore less commonly examined due to the effort cost involved. Interestingly, the spatially resolved physiomarker distribution in FIG. 7B displays a global maximum near the aortic arch as well as multiple pockets of instability N_{ω,sp}>0 along the descending aorta. This demonstrates that abnormal aortic dilatation is not necessarily confined near the SOV and MAA. More comprehensive imaging analysis motivated by predictive physiomarker distributions may help detect “silent-until-rupture” aneurysms that evade screening at common sites.

Aneurysm Development in Normal Patients with Respect to Age

Although age is not a direct input into the eigenvalue analysis that yields the stability parameter N_{ω,sp}, many physiological properties vary systematically with age. For instance, both aortic diameter and wall stiffness are known to increase naturally in older, healthy subjects. Less is known about how these age related variations affect aneurysm formation and growth. Important trends are discussed below.

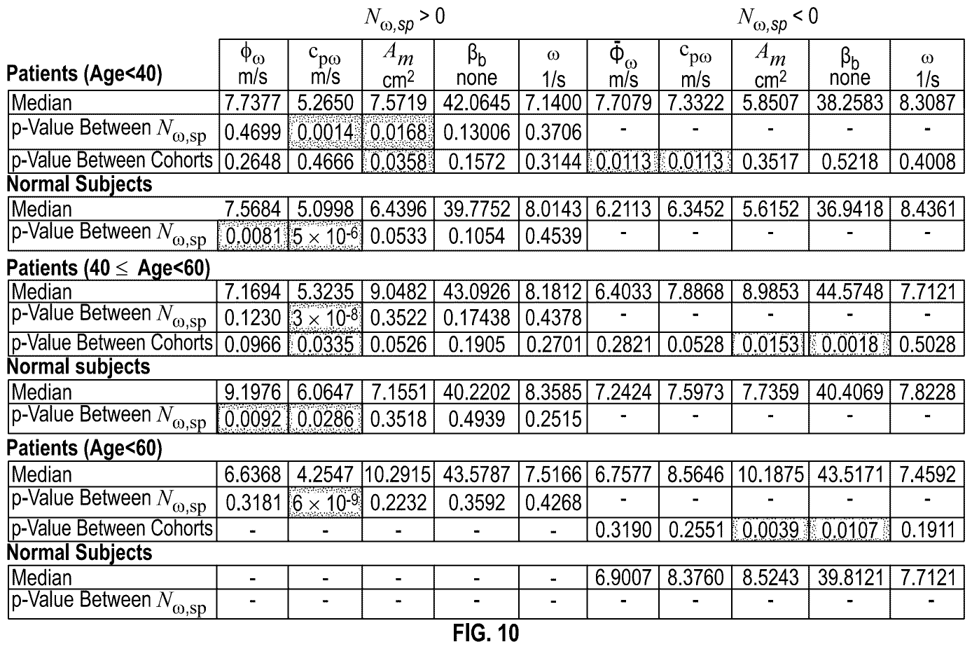

Table 4 in FIG. 10 shows the breakdown of physiological properties that make up the physiomarker N_{ω,sp}. This comparison occurs across three age groups. In the normal subject cohort, an unstable flow condition N_{ω,sp}>0 is induced on average by two significant factors relative to the stable N_{ω,sp}≤0 normal subjects−the larger pressure gradient φ̅ω that causes blood flow oscillatory acceleration as well as the smaller pulse wave velocity c_{pw}. N_{ω,sp} becomes positive primarily because N_{ω,clin} (eqn. 28) value increases for larger φ̅_{ω} and smaller c_{pw}, while N_{ω,threshold} decreases for smaller c_{pw}.

For the youngest age group (Age<40), the dominant factor is larger (p=0.0024) pressure gradient φ̅_{ω}. That is, normal subjects with unstable physiomarker N_{ω,sp}>0 exhibit larger φ̅_{ω} compared to normal subjects with a stable physiomarker N_{ω,sp}≤0 in a one-tailed Wilcoxon rank sum test. The role of greater blood flow acceleration is well established as a qualitative marker in aneurysm development; hypertension is well acknowledged as a risk factor in aneurysm formation and growth and has been implicated in modulating the morphology of unstable aneurysms.

In the middle age group (40≤Age<60), the pressure gradient φ̅_{ω} is likewise significantly higher for unstable aortic flow N_{ω,sp}>0. The second factor that appears is smaller (p=0.034) pulse wave velocity c_{pw}, which indicates lower aortic stiffness. Compliant aortic walls distend farther and can sustain more unstable flutter modes under the same pressure gradient compared to stiffer aortas characterized by higher c_{pw}. Thus, the natural stiffening of the aorta with age for healthy subjects serves to protect against further dilatation. This explains why the oldest age group (Age≥60) has no normal subjects exhibiting N_{ω,sp}>0.

Aneurysm Development in Patients with Respect to Age

In the patient cohort (see Table 4 in FIG. 10), flutter instability physiomarker N_{ω,sp}>0 is mainly driven by a smaller pulse wave velocity c_{pw} compared to patients experiencing stable flow N_{ω,sp}≤0. In the youngest age group (Age<40), we observe that aortic area Aₘ is likewise significantly higher for patients with positive physiomarker values. This matches clinical observations of increased dilatation risk with larger aneurysm size. As before, N_{ω,sp} becomes positive primarily because N_{ω,clin} (eqn. 28) increases and N_{ω,threshold} decreases for smaller c_{pw}. Larger aortic area also leads to increased N_{ω,clin} and, to a lesser extent, increased N_{ω,threshold}, such that the stability parameter (eqn. 28) increases overall.

In every age group for the patient cohort, the median pulse wave velocity is significantly lower for patients with unstable aortic flows N_{ω,sp}>0 compared to stable patients N_{ω,sp}≤0. This suggests that greater wall distensibility plays a dominant role in facilitating growth of larger, developed aneurysms in the patient cohort. Permanent dilatation occurs when the aortic wall weakens and becomes less stiff. Such a process can form a self-perpetuating cycle, since thinning of the intimal and medial layers during aneurysm expansion increases aortic distensibility, which supports further dilation by increasing aortic wall susceptibility to unstable flutter modes.

A summary of clinical observations on how aneurysm distensibility evolves during disease progression is discussed. As noted earlier, the aortic wall degrades due to elastin and smooth muscle loss through aneurysm enlargement. Hereafter, collagen deposition either stiffens the aortic wall (no further growth; Type 1) or wall weakens due to lack of collagen deposition, wall inflammation, and/or adipocyte accumulation (Type 2). The latter can lead to eventual dissection or rupture.

The disclosed physiomarker 22 provides support for these clinically observed pathways. Patients who exhibit stable flows N_{ω,sp}≤0 have significantly larger pulse wave velocities and therefore fall within the Type 1 “stiff” aneurysm group. On the other hand, every patient age group with unstable aortic flows N_{ω,sp}>0 has significantly lower pulse wave velocity than stable patients in the same age group. Thus, patients whose compliant aortic walls fail to respond and lay down collagen remain vulnerable to growth driven by the flutter instability. This indicates that unstable patients possess Type 2 “soft/at-risk” aneurysms.

### Cross Cohort Comparisons of Aneurysm Drivers

Next, different physiological properties driving aneurysm growth are compared between the normal subject cohort and the patient cohort.

In the youngest age group (Age<40), the stable N_{ω,sp}≤0 patient cohort exhibits a significantly larger median pulse wave velocity than stable normal subjects. This further reinforces the clinical observation that the branch of aneurysm progression toward the stable Type 1 aneurysm is marked by stiffening of the aortic wall that prevents additional dilatation. Similarly, the unstable N_{ω,sp}>0 patient cohort exhibits a significantly smaller median pulse wave velocity than stable (p=5×10⁻⁴) normal subjects. Thus, unstable patients comprise the second trajectory of aneurysm development—the Type 2 aneurysm group for which increased wall distensibility triggers further growth.

As the age of normal subjects increases through the three defined groups, the pulse wave velocity of stable normal subjects N_{ω,sp}≤0 increases significantly from (Age<40) to (40≥Age<60) with (p=0.0011), as well as from (40≥Age<60) to (Age≥60) with (p=0.0011). This reflects the natural stiffening of the aorta with age and also serves to protect against unstable flutter. However, the median pulse wave velocity of the youngest (Age<40) stable normal subject cohort is still significantly larger than that of unstable patients of any age group. The Type 2 progression of aortic aneurysms therefore marks a diseased state in which distensibility increases abnormally above reference, healthy values due to wall remodeling. This disease trajectory is especially prominent in the oldest age group (Age≥60), where the pulse wave velocity for the unstable patient cohort is significantly lower than that of the stable normal subject cohort (p=4×10⁻⁷). Natural stiffening of the aorta has entirely failed to kick in for the unstable patient cohort and is replaced by aneurysmal weakening of the wall. Thus, the trends obtained for the ab-initio physiomarker 22 are in good agreement with observed tissue biology during aneurysm development and it provides a quantitative prediction of anticipated growth.

The physiomarker 22 trends also elucidate other physiological drivers that contribute to abnormal aortic dilatation. For instance, the initial growth of aneurysms in normal subjects is driven mainly by a significantly larger pressure gradient φ̅_{ω} for (Age<40) and (40≥Age<60). Without the associated wall stiffening to constrain these unstable modes, due to a failure to remodel or insufficient response time relative to growth progression, abnormal aortic dilatation occurs. Meanwhile, the abnormal growth of aneurysm in the patient cohort for all age groups is driven primarily by lower pulse wave velocity, as we have already examined in depth. Thus, the fundamental physiology responsible for aneurysm progression varies significantly depending on whether the subject is in an earlier or later stage of the disease. Different treatment options and drug targets would then be necessary to address the root cause of abnormal growth for each patient depending on the dominant physiological property associated with or triggering the flutter instability. Quantitatively, this can be defined by measuring the sensitivity of N_{ω} to factors like pressure gradient φ̅_{ω} associated with blood oscillatory acceleration and pulse wave velocity c_{pw}. For instance, if reducing φ̅_{ω} to a manageable level would bring the physiomarker N_{ω} below 0, indicating stable flow, then blood pressure management may be the preferred course of treatment for a patient.

Finally, we note that the median aortic area Aₘ and oscillatory wall shear coefficient β_{b} are both significantly larger for stable patients compared to stable normal subjects in the age groups (40≥Age<60) to (Age≥60). In the same age groups however, these two physiological properties are not significantly different between stable patients and unstable patients, nor for stable normal subjects and unstable normal subjects. This suggests that larger Aₘ and β_{b} accompany disease progression and may differentiate between subjects who have already experienced aortic dilatation, but not necessarily drive further, abnormal growth on a consistent basis.

In existing literature, many ambiguous observations surround each of the individual physical properties examined in Table 4 in FIG. 10. For instance, a definite relationship between hypertension and aneurysm growth is not apparent, especially since patients without hypertension can likewise experience both aneurysm growth and rupture. High blood pressure is often interpreted as the mechanism driving increased shear stress along the aortic walls, but both high wall shear stress, low wall shear stress, and the spatio-temporal heterogeneity of wall shear stress have been implicated in wall remodeling and aneurysm growth. Similarly, larger aortic size is known to correlate with increasing risk of rupture, but it is unclear why this is the case.

The physiomarker 22 explains not just how these properties trend at the cohort level, but also reveals the mechanism of how they interact explicitly in each patient. It clarifies the role of each physical property in driving the flutter type instability and delineates the threshold which separates stable aneurysms from unstable growth. These physiological properties cannot be used to predict abnormal dilatation on their own without knowing their relative, quantitative role in driving or inhibiting aneurysm growth for each patient—this, we propose, is the key problem resolved by the physiomarker N_{ω,sp}>0.

The prediction of flutter is a linear approximation, in the sense that the flutter and stability parameters measures whether flutter occurs given the patient's current imaged physiological properties. It does not account for changes in physiological properties like blood pressure, aortic stiffness, aortic size, etc. from year to year. In essence, observed flutter now is clinically indicative of abnormal growth in the future. This can be ameliorated by more frequent surveillance such as annual or bi-annual imaging for at-risk patients identified via N_{ω,sp}>0.

Lastly, the disclosed technology provides a linear stability analysis of a 1D blood vessel model. The immediate advantage is that the problem becomes tractable and yields a closed form solution. However, nonlinear damping or instability inducing effects may become important in certain flow conditions. Similarly, the asymmetry, curvature, and branching of the aortic geometry may also play a significant role.

### CONCLUSION

The disclosed technology provides an instability-driven growth mechanism of aortic aneurysms from first principles through a linear stability analysis of flow through an elastic blood vessel 10. The perturbation equations around the base flow gives a dispersion relation between the temporal growth rate of each flutter mode and its wave number. Floquet theory is used to account for the parametric effect of the heartbeat frequency—essentially, the oscillatory blood flow waveform.

The important parameters determining the onset of unstable flutter—including viscosity, vessel diameter, pressure gradient that drives acceleration, etc.—are collected in a single dimensionless number, which we call the flutter parameter. This parameter tracks the transition of the system to the flutter type instability. If the flutter parameter at a local cross-section of the blood vessel exceeds an analytically derived threshold, the growth of perturbation modes may trigger the cross-sectional area of the blood vessel to dilate abnormally. An aneurysm will form or grow at the site. Otherwise, perturbation amplitudes decay in time, and the location remains stable to this flutter mechanism.

Through follow-up analysis in a group of patients with suspected aortopathy, it has been shown that the proposed stability parameter may be used as a physiomarker to forecast aneurysm growth. The only input to calculate the parameter for each patient was a baseline 4D flow magnetic resonance imaging scan taken during the initial visit. This physiomarker predicts abnormal aortic growth and/or surgical intervention at clinical follow-up with high accuracy, specificity, and sensitivity.

This ab-initio physiomarker 22 is a predictive diagnostic tool for aneurysm development. It captures the observed qualitative population trends in subjects and clarifies the qualitative growth modes of nascent aortic dilation vs. the evolution of large, developed aneurysms. Here, a full derivation of the stability parameter is presented, in addition to testing its potential for diagnostic capability and its contextualization as a fundamental mechanistic precursor to aneurysm formation and growth.

Other systems, methods, features and advantages will be, or will become, apparent to one with skill in the art upon examination of the figures and detailed description. It is intended that all such additional systems, methods, features and advantages be included within this description, be within the scope of the disclosure, and be protected by the following claims.

What is claimed is:

1. A method for deriving an aneurysm physiomarker, comprising:
analyzing a single 4D flow magnetic resonance imaging (MRI) scan associated with a blood vessel of a patient, wherein the MRI scan is taken at an initial time point;
determining a critical threshold beyond which an area of the blood vessel in the MRI scan fluctuates unboundedly under infinitesimal perturbations;
selecting a base flow comprising a periodic limit cycle following a pulsatile waveform of blood pressure over a cardiac cycle associated with the blood vessel in the MRI scan; and
generating a flutter parameter for describing an onset of an instability triggering fluttering of a vessel wall of the blood vessel in the MRI scan, wherein generating the flutter parameter is based on at least the analyzing of the single 4D flow MRI scan, determining the critical threshold, and the selecting the base flow.

2. The method of claim 1, further comprising:
resolving effect of the infinitesimal perturbations at higher order frequencies via Floquet theory.

3. The method of claim 1, further comprising:
capturing physical interaction of known physiological factors in progression and development of an aneurysm associated with the blood vessel in the MRI scan.

4. The method of claim 3, wherein the known physiological factors is at least one of blood pressure, aortic size, wall shear stress, and pulse wave velocity.

5. An aneurysm physiomarker, comprising:
a flutter parameter for describing an onset of an instability triggering fluttering of a vessel wall of a blood vessel in a single 4D flow magnetic resonance imaging (MRI) scan associated with a blood vessel of a patient, wherein the flutter parameter is generated based on at least
analyzing the single 4D flow MRI scan associated with the blood vessel of the patient, wherein the MRI scan is taken at an initial time point,
determining a critical threshold beyond which an area of the blood vessel in the MRI scan fluctuates unboundedly under infinitesimal perturbations, and
selecting a base flow comprising a periodic limit cycle following a pulsatile waveform of blood pressure over a cardiac cycle associated with the blood vessel in the MRI scan.

6. The aneurysm physiomarker of claim 5, wherein the flutter parameter is generated based further on at least
resolving effect of the infinitesimal perturbations at higher order frequencies via Floquet theory.

7. The aneurysm physiomarker of claim 5, wherein the flutter parameter is generated based further on at least
capturing physical interaction of known physiological factors in progression and development of an aneurysm associated with the blood vessel in the MRI scan.

8. The aneurysm physiomarker of claim 7, wherein the known physiological factors is at least one of blood pressure, aortic size, wall shear stress, and pulse wave velocity.
